(19) United States
(12) Patent Application Publication
(10) Pub. No.: US 2026/0306620 A1
(43) Pub. Date: Oct. 8, 2026

(54) TUMOR NEOANTIGEN VACCINE SYSTEM SPECIFICALLY MARKING HETEROLOGOUS PROTEIN, AND USE THEREOF

(71) Applicant: CHINA PHARMACEUTICAL UNIVERSITY, Jiangsu (CN)

(72) Inventors: Yong YANG, Jiangsu (CN); Wenguang WANG, Jiangsu (CN)

(73) Assignee: CHINA PHARMACEUTICAL UNIVERSITY, Jiangsu (CN)

(21) Appl. No.: 19/643,770

(22) Filed: Apr. 9, 2026

Related U.S. Application Data
(63) Continuation of application No. PCT/CN2023/125912, filed on Oct. 23, 2023.

(30) Foreign Application Priority Data
Oct. 17, 2023 (CN) 202311338909.3

(51) Int. Cl. A61K 39/29; A61K 9/00; A61K 35/768; A61K 39/00; A61P 35/00; C12N 7/00

(52) U.S. Cl. CPC A61K 39/292; A61K 9/0019; A61K 35/768; A61P 35/00; C12N 7/00; A61K 2039/53; A61K 2039/54; C12N 2710/24132; C12N 2710/24151; C12N 2730/10134

(57) ABSTRACT

The present invention provides a tumor neoantigen vaccine system specifically marking a heterologous protein, and a use thereof. The vaccine system is composed of a heterologous protein mRNA vaccine and a recombinant oncolytic virus; the heterologous protein mRNA vaccine is used to induce an organism to generate heterologous protein-specific memory T cells, and the recombinant oncolytic virus is used to promote a tumor to express microorganism-derived tumor neoantigens. When pre-immunized memory T cells detect these marked specific neoantigens, the tumor expressing the neoantigens is quickly activated and killed, the release of tumor autoantigens is promoted, dendritic cells phagocytize antigens to further initiate an anti-tumor response, and a systemic anti-tumor immune effect is activated by means of antigenic epitope spreading.

### CROSS-REFERENCE TO RELATED APPLICATION

The present application is a continuation of international application of PCT application serial no. PCT/CN2023/125912, filed on Oct. 23, 2023, which claims the benefit of priority of Chinese application no. 202311338909.3, filed on Oct. 17, 2023. The entirety of each of the above-mentioned patent applications is hereby incorporated by reference herein and made a part of this specification.

### REFERENCE TO A SEQUENCE LISTING

The instant application contains a Sequencing Listing which has been submitted electronically in XML file and is hereby incorporated by reference in its entirety. Said XML copy, created on Mar. 26, 2026, is named 165419_SEQUENCELISTING and is 11,496 bytes in size.

### TECHNICAL FIELD

The present invention relates to an anti-tumor vaccine system, and in particular to a tumor neoantigen vaccine system specifically marking a heterologous protein, and also to a use of the above-mentioned vaccine system.

### BACKGROUND OF RELATED ART

As a milestone event in the history of tumor treatment, immunotherapy has greatly changed the landscape of clinical oncology therapy. Due to their unique tumor-targeting property, tumor neoantigen vaccines have become a research hotspot in immunotherapy. Neoantigens, also known as tumor-specific antigens, are expressed only in tumors but not in normal tissues. By activating the patient's immune system, neoantigens can induce tumor-specific T cells to inhibit tumor growth. Although tumor neoantigens have advantages such as high tumor specificity compared with conventional tumor-associated antigens, multiple clinical trials conducted both domestically and internationally have demonstrated that tumor neoantigen vaccines suffer from poor response rates in the treatment of various solid tumors. For example, the personalized tumor neoantigen vaccine RO7198457 developed by BioNTech achieved an objective response rate of only 8% in the treatment of multiple solid tumors, including melanoma, breast cancer, and colorectal cancer. Existing tumor neoantigens still suffer from the following problems: low immunogenicity and highly heterogeneous expression, as well as suppression of the therapeutic response to tumor neoantigen vaccines by the tumor immune microenvironment (TIME).

Oncolytic viruses are a novel therapy for tumor treatment. Oncolytic viruses are a class of natural or genetically engineered viruses that selectively infect and kill tumor cells without affecting normal cells. However, oncolytic viruses have significant limitations in tumor therapy. Since the immunogenicity of tumor antigens is often lower than that of viral vector-derived proteins, the organism may generate a strong antiviral immune response, thereby inhibiting viral replication and oncolysis, but failing to produce a potent anti-tumor therapeutic effect. Although oncolytic viruses expressing immune checkpoint inhibitors such as PD-1, dendritic cell (DC) activation factors such as GM-CSF, and cytokines such as IL-2 can enhance the anti-tumor immune effect of oncolytic viruses to some extent, they still rely on de novo initiation from tumor-derived antigens and therefore remain limited by the organism immune system's inhibitory effect on the virus, failing to produce a significant anti-tumor therapeutic effect.

### SUMMARY OF THE INVENTION

Objective: An objective of the present invention is to provide an efficient tumor neoantigen vaccine system specifically marking a heterologous protein that uses memory T cells generated by an mRNA vaccine to recognize and kill tumor cells precisely marked by the neoantigen derived from the heterologous protein, thereby activating systemic anti-tumor immune effects. The present invention also provides a use of the vaccine system.

Technical Solution: The present invention provides a tumor neoantigen vaccine system specifically marking a heterologous protein, the vaccine system is composed of a heterologous protein mRNA vaccine and a recombinant oncolytic virus. The heterologous protein mRNA vaccine generates specific memory T cells that precisely mark neoantigens, and the recombinant oncolytic virus is used to precisely mark tumor cells with strong immunogenic neoantigens.

Preferably, the heterologous protein is one or more selected from Hepatitis B virus-derived protein (HBsAg), human papillomavirus-derived protein (E6, E7), EBV-derived protein (PP65), influenza virus-derived protein (MP), tetanus toxoid protein (TT), Staphylococcus aureus-derived protein (C2), or flagellin protein of Salmonella typhi.

Preferably, the oncolytic virus is one or more of vaccinia virus, herpes simplex virus, or adenovirus.

Preferably, the heterologous protein mRNA vaccine is prepared by constructing, synthesizing, and amplifying a recombinant plasmid of the mRNA vaccine, followed by encapsulating the recombinant plasmid of the mRNA vaccine with lipid nanoparticles (LNP).

Preferably, the recombinant oncolytic virus is prepared by constructing a recombinant oncolytic virus expressing the neoantigen, followed by plaque purification and amplification purification.

Use of the vaccine system in the preparation of a tumor immunotherapy drug is also provided.

Preferably, the vaccine system is used to enhance tumor immunotherapy; the heterologous protein mRNA vaccine is used to induce an organism to generate heterologous protein-specific memory T cells, and the recombinant oncolytic virus is used to promote a tumor to express microorganism-derived tumor neoantigens. When pre-immunized memory T cells detect these marked specific neoantigens, the tumor expressing the neoantigens is quickly activated and killed, the release of tumor autoantigens is promoted, dendritic cells (DC) phagocytize antigens to further initiate an anti-tumor response, and a systemic anti-tumor immune effect is activated.

Preferably, an administration sequence involves first administering the heterologous protein mRNA vaccine, followed by administration of the recombinant oncolytic virus, or administration of the heterologous protein mRNA vaccine and the recombinant oncolytic virus simultaneously.

Preferably, the heterologous protein mRNA vaccine may be administered by intramuscular injection, intranasal administration, or intravenous injection to induce immune memory of the organism, and the recombinant oncolytic virus may be administered intratumorally or intravenously to precisely mark tumor cells with strong immunogenic neoantigens.

Preferably, the tumor is a solid tumor or a solid tumor with concomitant metastasis, including melanoma, colorectal cancer, breast cancer, liver cancer, glioblastoma, cholangiocarcinoma, or pancreatic cancer.

Invention principle: To solve the problems of low immunogenicity, tumor heterogeneity (spatial and temporal differences in neoantigen expression within the tumor), and limitations in the tumor immune microenvironment (TIME) of the neoantigens that lead to poor therapeutic response to neoantigen vaccines, the present invention constructs a tumor neoantigen vaccine system specifically marking a heterologous protein consisting of a heterologous protein mRNA vaccine and a recombinant oncolytic virus. On one hand, by constructing the tumor neoantigen derived from the heterologous protein with strong immunogenicity, the system solves the problems of low immunogenicity and heterogeneous expression identified in current neoantigen derived from tumor gene mutation. On the other hand, the heterologous protein mRNA vaccine stimulates the organism to produce neoantigen-specific memory T cells that break through the limitations in TIME, thereby activating anti-tumor immune effects through a two-pronged approach. The present invention aims to improve the therapeutic response rate of neoantigen vaccine by independently constructing highly immunogenic tumor neoantigen and inducing neoantigen-specific memory T cells that break through the limitations in TIME. The present invention solves the following three problems in the prior art:
(1) Solving the problem of low immunogenicity and heterogeneous expression of existing tumor neoantigens: The use of a gene-defective oncolytic virus specifically marks a tumor cell with a neoantigen derived from the heterologous protein that is highly safe and strongly immunogenic. The low immunogenicity and heterogeneous expression of the tumor mutation-derived neoantigen are key bottlenecks leading to poor response rate for the neoantigen vaccine in solid tumors, which need to be urgently solved. The present invention leverages the characteristic of the gene-defective oncolytic virus that replicates only in tumors, to specifically mark a tumor cell with a neoantigen derived from the heterologous protein that is highly safe and strongly immunogenic, thereby solving the problem of low immunogenicity and heterogeneous expression of existing mutation-derived neoantigens. More importantly, the heterologous protein constructed in the present invention has known strong T-cell epitopes, eliminating the need for time-consuming and costly processes such as neoantigen sequencing and screening, which allows for rapid use in tumor treatment.
(2) Solving the problem of suppression of the therapeutic response of tumor neoantigen vaccines by TIME: The mRNA vaccine induces the organism to produce neoantigen-specific memory T cells (which are not limited by TIME) and specifically marks neoantigen, enabling fast and efficient tumor killing and activation of anti-tumor immune effects. Even if tumor mutation-derived neoantigen-specific T cells are generated, TIME may prevent these neoantigen-specific T cells from recognizing and killing tumors by exhausting them. In contrast, immune memory T cells, especially those specific to heterologous proteins, have a low activation threshold when recognizing antigens and are not limited by TIME. The present invention takes advantage of the characteristic of heterologous protein-specific memory T cells that are not limited by TIME, and specifically marked neoantigen derived from heterologous protein to rapidly and efficiently kill tumors, release tumor-derived antigens (epitope spreading), thereby further activating anti-tumor immune effects and improving the therapeutic response rate of neoantigen vaccine.
(3) Solving the scientific problem of poor anti-tumor immune effects induced by oncolytic viruses: Theoretically, oncolytic viruses can eliminate tumors by lysing tumor cells, releasing tumor antigens, and activating anti-tumor immune response. However, since the immunogenicity of tumor antigens is often lower than that of the virus, which leads to a stronger anti-virus immune response of the organism, and further limits the virus' oncolytic activity. This results in less tumor lysis and antigen release, leading to poor anti-tumor immune effects. Oncolytic viruses expressing PD-L1, GM-CSF, neoantigens, etc. can enhance anti-tumor immune responses to some extent but still depend on the immunogenic initiation of tumor antigens. Therefore, they are still limited by organism's immune suppression of the virus. To solve the aforesaid problem, on one hand, the present invention enhances tumor immunogenicity by delivering the neoantigen derived from heterologous protein; on the other hand, the present invention pre-induces the organism to produce a large number of neoantigen-specific memory T cells by using the mRNA-H vaccine. These heterologous protein-specific memory T cells can rapidly and efficiently kill tumors expressing neoantigens, release tumor antigens, and activate anti-tumor immune effects. Therefore, the present invention also solves the problem of poor anti-tumor immune effects caused by host immune limitations on oncolytic virus.

Since most microbial-derived proteins, such as human papillomavirus-derived proteins E6 and E7, EBV-derived protein PP65, influenza virus-derived protein, tetanus toxoid protein, Staphylococcus aureus-derived protein, or flagellin protein of Salmonella typhi, are all heterologous microbial proteins that have known strong immunogenic epitopes, and exhibit stronger immunogenicity than neoantigens derived from single tumor gene mutations. Therefore, using mRNA vaccines corresponding to these proteins (to generate neoantigen-specific memory T cells derived from these heterologous proteins and break through the limitations in TIME) combined with oncolytic viruses to precisely mark tumor cells with these neoantigens (solving the problems of low immunogenicity and heterogeneous expression in tumor mutation-derived neoantigens) can achieve therapeutic effects similar to those of marking the HBsAg-derived neoantigen. Similar to vaccinia virus, the oncolytic virus can specifically infect tumor cells without infecting surrounding normal cells. Therefore, using oncolytic viruses such as herpes simplex virus or adenovirus as delivery vectors to precisely mark neoantigens derived from heterologous proteins can also achieve effects similar to those of oncolytic vaccinia virus, as they can precisely mark tumor cells with neoantigens derived from heterologous proteins.

While using the oncolytic virus to precisely mark the tumor cell with strong immunogenic heterologous protein and constructing strong immunogenic tumor neoantigen to solve the problems of low immunogenicity and heterogeneous expression of existing tumor neoantigens, the vaccine system of the present invention also uses the mRNA vaccine to pre-stimulate the organism to generate neoantigen-specific T cells (not limited by TIME). By leveraging the characteristics of precisely marked strong immunogenic neoantigen and the memory T cells not limited by TIME, the system can rapidly and efficiently kill solid tumors, thereby activating anti-tumor immune effects through a two-pronged approach, and improving the therapeutic response rate of tumor vaccine. Furthermore, memory T cells generated by the mRNA vaccine efficiently solve the problem that oncolytic virus cannot activate a strong anti-tumor immune response caused by host immune limitations. Before the immune system of the organism can recognize and eliminate the tumor, they can quickly recognize and kill tumor cells and activate anti-tumor immune effects. The recombinant oncolytic virus is a gene-defective vaccinia virus that replicates only in tumors, not in normal tissues, ensuring the precise delivery of heterologous protein.

Beneficial effects: compared with the prior art, the present invention has the following advantages:
(1) The heterologous protein mRNA vaccine in the present invention stimulates the organism to generate a stronger memory T cell response: Compared with the commercially available recombinant protein vaccines, the mRNA-H group (i.e., the mRNA vaccine for heterologous protein HBsAg) showed a 150-fold increase in IFN-γ⁺ cells and at least a 25-fold increase in IFN-γ⁺CD8⁺ T cells in the mouse spleen cells after being re-stimulated with the antigen. This effectively overcomes the tumor immune suppressive microenvironment and improves the therapeutic response rate of neoantigen vaccines. The extremely high response rate combined with the recombinant oncolytic virus achieves more precise and efficient therapeutic effects.
(2) In the mouse B16F10 melanoma experiment, tumor growth in the treatment group was significantly inhibited. Moreover, flow cytometry results showed at least 10-fold and 4-fold increase in tumor-infiltrating CD8⁺ T cells and CD4⁺ T cells in the treatment group, respectively, and effector memory T cells (TEM) in the spleen also increased significantly.
(3) In three different levels of solid tumors, namely, B16F10-OVA melanoma, 4T1 breast cancer (low immunogenicity, highly malignant, and prone to spontaneous metastasis), and CT26 (immunologically cold tumor), the recombinant oncolytic virus VV-H of the present invention successfully marked neoantigen derived from heterologous protein and activated its specific memory T cells, thereby resulting in synergistic effects and significantly different therapeutic effects on solid tumors.

### BRIEF DESCRIPTION OF THE DRAWINGS

FIG. 1 is a flowchart and explanation of the background technical process.

FIG. 2 is a flowchart of use of a vaccine system of the present invention in tumor immunotherapy, where a-b refer to the immunization of mice with mRNA-H vaccine (intramuscular injection) to stimulate an organism to produce HBsAg memory T cells, c refers to use of VV-H (intratumoral injection) to specifically mark the tumor with an HBsAg neoantigen, d refers to the activation of memory T cells by an HBsAg neoantigen, rapidly killing a tumor and releasing tumor antoantigens, e refers to epitope spreading, and f refers to the generation of systemic anti-tumor immune effects.

FIG. 3 is a pUC-HBsAg recombinant plasmid map.

FIG. 4 shows an average particle size, surface potential, and mean dispersion coefficient of LNP HBsAg mRNA.

FIG. 5 shows a quantification of serum HBsAb 7 days after first (a) and second (b) immunization with an mRNA-H vaccine.

FIGS. 6A to 6D show characterization of HBsAg memory T cells in a spleen after HBsAg vaccine immunization; where FIGS. 6A and 6B refer to IFN-γ⁺ cells after stimulation with the ELISPOT quantitative HBsAg peptide library; and FIGS. 6C and 6D refer to a flow cytometry gating strategy and quantification of IFN-γ⁺CD8⁺ T cells after restimulation with the HBsAg peptide library.

FIG. 7 shows efficacy study of an mRNA-H vaccine in preventing B16-HBsAg tumor, where a shows tumor volume monitoring (n=5); b shows body weight monitoring of tumor-bearing mice during treatment (n=5); c shows survival rate statistics of mice after different vaccine immunizations (n=6), HepB-CHO represents a commercially available recombinant HBsAg hepatitis B vaccine.

FIG. 8 shows immunogenicity evaluation of the neoantigen HBsAg, including tumor volume and body weight monitoring of B16-HBsAg, B16-OVA, and B16F10 tumor model groups (n=10).

FIG. 9 shows evaluation of epitope spreading of the neoantigen HBsAg, with ELISPOT quantification of IFN-γ⁺ cells after stimulation with a melanoma peptide library in B16-HBsAg tumor-bearing mice.

FIG. 10 shows a plasmid map and gene construction process of an oncolytic virus, where A is a vaccinia virus plasmid map, and B shows gene construction process.

FIG. 11 shows red fluorescence images of Hela-s3 cells during recombinant virus screening and culture.

FIG. 12 shows PCR identification of recombinant oncolytic vaccinia virus expressing a target genes, with HBsAg and GM-CSF gene primers, where the left image shows HBsAg, and the right image shows GM-CSF.

FIG. 13 is a bar chart showing the ability of VV-H to induce HBsAg and GM-CSF expression in B16F10 tumor cells using an ELISA method.

FIG. 14 shows the expression of TK gene identified by a recombinant oncolytic vaccinia virus gDNA after plaque purification.

FIG. 15 shows an HBsAg content in Hela-s3 tumor cells after 24 h, 48 h, and 72 h of VV-H infection under different MOIs using an ELISA method.

FIG. 16 shows a treatment protocol for a vaccine system of the present invention to activate anti-tumor immune effects, where s.c. refers to subcutaneous injection and i.t. refers to intratumoral injection.

FIGS. 17A to 17E show evaluation of therapeutic effects of VV-H-activated HBsAg memory T cells in B16F10 melanoma, where FIG. 17A shows tumor volume monitoring, FIG. 17B shows body weight monitoring of tumor-bearing mice, FIGS. 17C and 17D show quantification of CD3⁺CD8⁺, CD3⁺CD4⁺ T cells in tumors (Gate: CD45+ cells), and FIG. 17E is a representative flow cytometry gating strategy.

FIGS. 18A to 18G show evaluation of therapeutic effects of VV-H-activated HBsAg memory T cells in B16-OVA melanoma, where FIGS. 18A to 18C show tumor volume and body weight monitoring, FIG. 18D shows a tumor status at a treatment endpoint, FIG. 18E shows tumor weight of tumor-bearing mice, and FIGS. 18F and 18G show quantification of CD3⁺CD8⁺, CD3⁺CD4⁺ T cells in tumors.

FIG. 19 shows evaluation of therapeutic effects of VV-H-activated HBsAg memory T cells combined with an immune checkpoint inhibitor PD-L1 in colorectal cancer (CT26), where a shows tumor volume monitoring, and b shows body weight monitoring of tumor-bearing mice.

FIGS. 20A to 20C show evaluation of therapeutic effects of VV-H-activated HBsAg memory T cells in breast cancer (4T1), where a shows tumor volume monitoring, b shows survival rate statistics, c shows recurrence rate statistics of tumor-bearing mice.

FIG. 21 shows immunofluorescence images of tumor expression of HBsAg induced by VV-H in vivo; tumors were excised after intratumoral injection of VV-H and subjected to HBsAg immunofluorescence staining (green for HBsAg) and VV (mCherry, the red fluorescence of the virus itself).

FIGS. 22A and 22B show safety evaluation after VV-H+ mRNA-H treatment, where a shows serum biochemical indicators after treatment, including alanine aminotransferase (ALT), total protein (TP), serum creatine kinase isoenzyme (CKI), and blood urea nitrogen (BUN), and b shows HE staining of major organs after treatment.

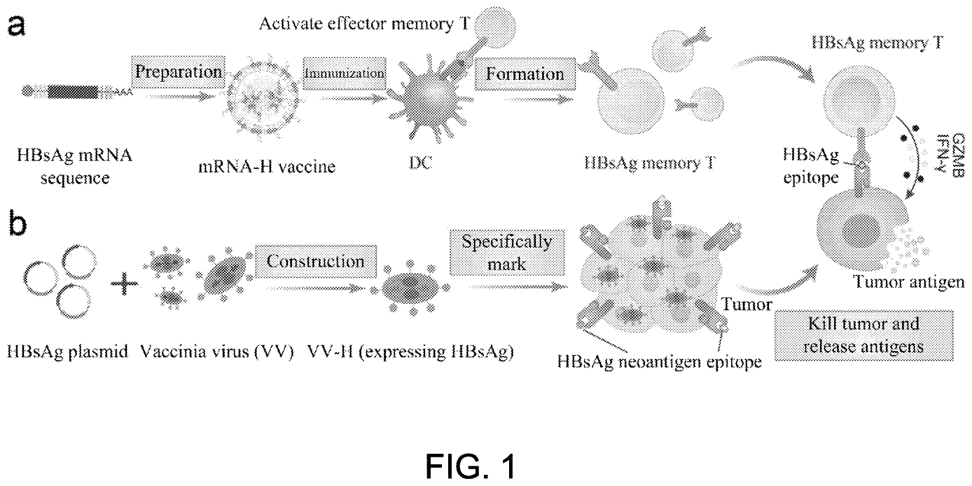

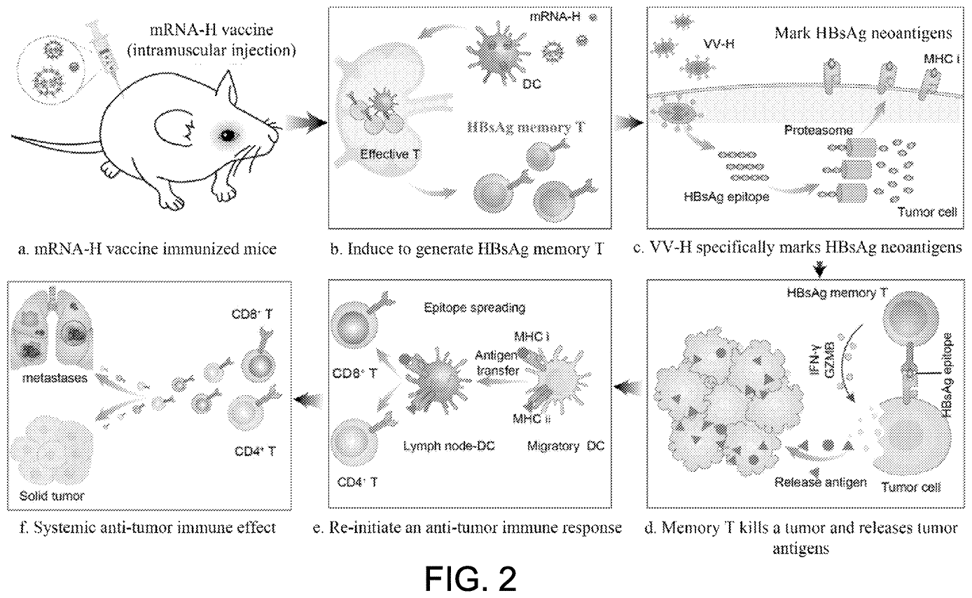

### DETAILED DESCRIPTION OF THE EMBODIMENTS

The technical solution of the present invention will be further described below with reference to the accompanying drawings.

### Example 1

Preparation of heterologous protein mRNA vaccine

1. Construction of mRNA-H:
(1) Construction, Synthesis and Amplification of Recombinant Plasmid for mRNA Vaccine

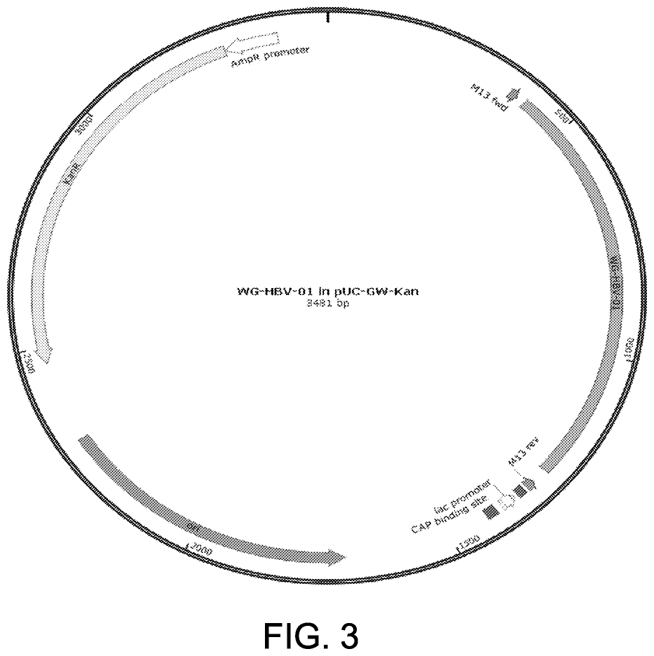

HBsAg, a T7 promoter, and UTR sequences were ligated into a vector to construct a pUC-HBsAg recombinant plasmid. A plasmid map is shown in FIG. 3. The plasmid was synthesized by Genewiz Company and Escherichia coli containing the recombinant plasmid was prepared. An appropriate amount of Escherichia coli bacterial solution containing the HBsAg plasmid was taken and incubated overnight in a 37° C. incubator. Three single bacterial colonies were picked and placed in centrifuge tubes containing 5 mL of culture medium, and cultured at 37° C. and 220 rpm for 14-18 h. The pHBsAg plasmid was extracted and quantified using a kit.

### (2) PCR, Purification and Identification of HBsAg Plasmid

A PCR reaction system was prepared and a PCR program was run. A 1% agarose gel was prepared, PCR products were mixed and three times the volume of Buffer DC were added and further mixed uniformly, then transferred to an adsorption column. The column was centrifuged at 12,000 rpm for 1 min at room temperature and a filtrate was discarded. Buffer WB was added to the adsorption column and allowed to stand for 2 min. The column was centrifuged at 12,000 rpm for 30 s at room temperature and a filtrate was discarded. Sterile water was added dropwise to a center of the adsorption column and centrifuged to collect a sample.

### (3) In Vitro Transcription (IVT) and Capping

An IVT reaction system was added and mixed uniformly, and then run in a PCR instrument at 37° C. for 2 h. DNase I was added, sterile water was added to the PCR product to supplement to 50 μL, mixed uniformly by pipetting, and incubated at 37° C. in a PCR instrument for 15 min to remove the DNA template. RNA cleanup binding buffer was added to the 100 μL of a sample, ethanol was then added and mixed uniformly by pipetting to obtain a solution; and the solution was transferred to a purification column and centrifuged at 12,000 rpm for 1 min at room temperature, and a filtrate was discarded. RNA cleanup wash buffer was added and centrifuged at 12,000 rpm for 1 min at room temperature. The previous step was repeated, and the column was transferred to an RNase-free centrifuge tube. An appropriate amount of sterile water was added, allowed to stand for 5 min, and centrifuged to collect a sample. Quantitative dilution was performed for agarose gradient gel electrophoresis.

2. Preparation of Heterologous Protein mRNA Vaccine

Each lipid component was mixed according to a molar ratio of Lipid/DSPC/cholesterol/PEG-DMG=50/10/38.5/1.5, and a 2.85 mg/mL lipid mixture was prepared by mixing Anhydrous ethanol with each lipid solution. Preparation of an aqueous solution containing mRNA: 100 μg of mRNA was diluted in 2 mL of 10 mM citrate-sodium citrate buffer to obtain an mRNA dilution. The above aqueous phase containing mRNA and the alcohol phase containing lipids were passed through a microfluidic device at a flow rate ratio of 3:1 to prepare mRNA LNP. After ultrafiltration and centrifugation, a particle size and zeta potential of the mRNA LNP were measured.

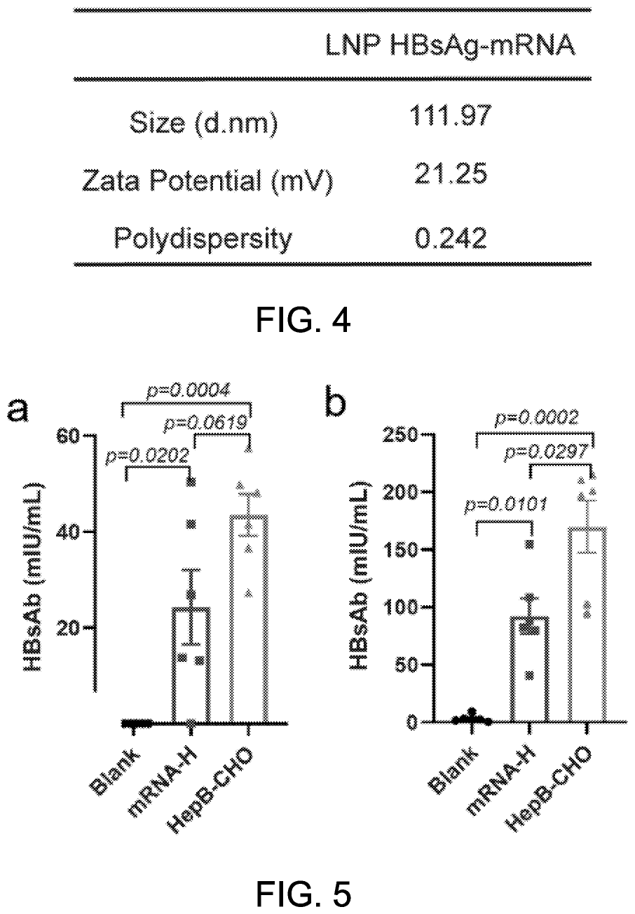

As shown in FIG. 4, the heterologous protein mRNA vaccine mRNA-H has suitable physical parameters, which are conducive to its entry of cells to express HBsAg. A plasmid nucleotide sequence of HBsAg mRNA is shown in SEQ ID NO. 1.

### Example 2

Detection of Anti-HBsAg Antibody (HBsAb) in Serum after Immunization with mRNA-H

The immunogenic effects of the prepared mRNA-H vaccine and the marketed HBsAg hepatitis B vaccine (HepB-CHO) were preliminarily evaluated by detecting HBsAb in serum. 6-week-old C57BL/6 mice were randomly divided into three groups: 1. Saline group; 2. mRNA-H vaccine group; and 3. HepB-CHO group. On Day 0 and Day 14 after grouping, the mice were immunized twice by intramuscular injection. On Day 7 following each immunization, the serum HBsAb level was measured.

As shown in FIG. 5, 7 days after the second immunization, the serum HBsAb level in the mRNA-H group was significantly higher than that in the Saline group (HBsAb≥10 mIU/mL is sufficient to prevent hepatitis B).

### Example 3

Detection of the Ability of mRNA-H Vaccine to Induce Splenic Memory T Cell Formation

ELISPOT assay: Mice were immunized using the method described in Example 3. 30 days after the final immunization, spleens were removed under sterile conditions and prepared into single-cell suspensions. The suspensions were centrifuged at 350 g for 5 min. Supernatant was discarded, 1 mL of red blood cell lysis buffer was added and lysed for 10 min, followed by addition of 3 mL of buffer to terminate red blood cell lysis. Cells were collected by centrifugation at 350 g for 5 min. 4 mL of RPMI-1640 medium was added to resuspend the cells, which were then counted and adjusted to 2×10⁶/mL. Activation of the pre-coated plate: 200 μL/well of RPMI-1640 medium (5% FBS) was added and allowed to stand for 10 min, and then removed. ELISPOT plating was performed as follows: Negative control group: 100 μL cells+5 μL PBS; HBsAg protein group: 100 μL cells+10 μL HBsAg epitope peptide library; and positive group: 100 μL cells+10 μL positive control stimulus. After addition, the plate was covered and incubated for 16-24 h. After incubation, spots were detected and statistically analyzed. In addition, the ability of memory T cells in the spleen to secrete IFN-γ after stimulation with the HBsAg epitope antigen library was verified using flow cytometry.

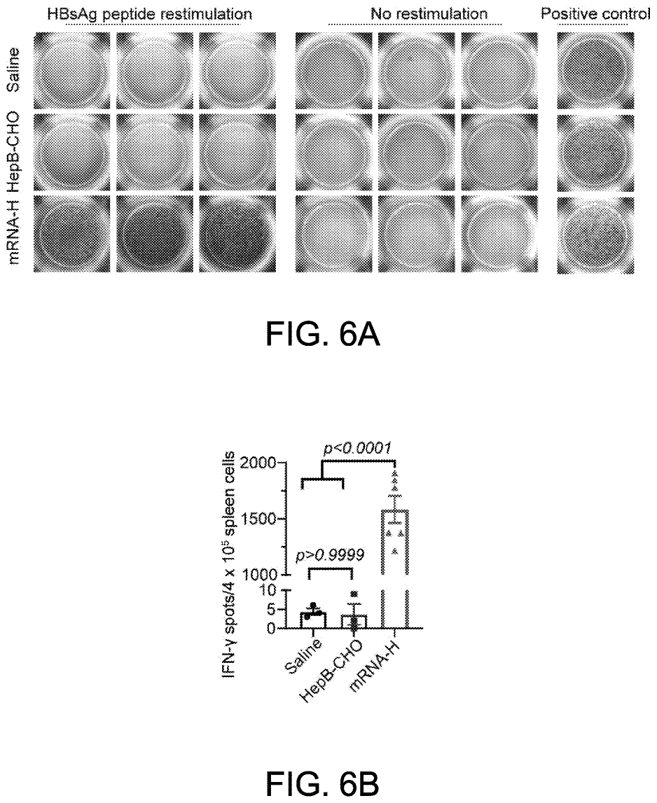

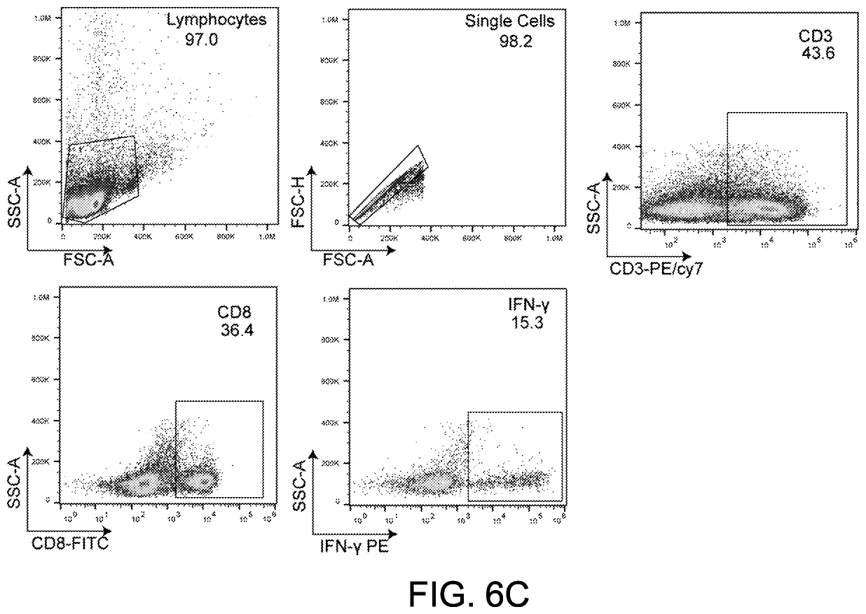

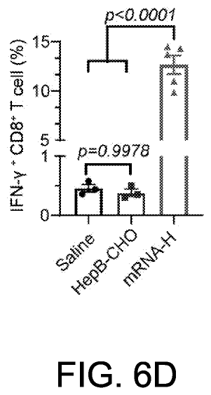

As shown in FIGS. 6A to 6D, compared with the recombinant HBsAg vaccine group, the mRNA-H vaccine produced at least 150-fold HBsAg-specific memory T cells, and IFN-γ⁺CD8⁺ T cells increased by at least 25-fold.

### Example 4

Evaluation of Therapeutic Effect of mRNA-H-Induced Memory T Cells in Preventing B16-HBsAg Tumors

6-week-old female C57BL/6 mice were randomly divided into three groups: 1. blank group; 2. mRNA-H group; and 3. HepB-CHO group. The mice were immunized according to the method described in Example 3. In addition, a B16F10 tumor expressing HBsAg (B16-HBsAg) was constructed using lentivirus. 30 days after the final immunization, the mice were inoculated with B16-HBsAg cells, the mice were observed for their conditions and a tumor volume was monitored. Moreover, repeated treatment was performed for survival analysis.

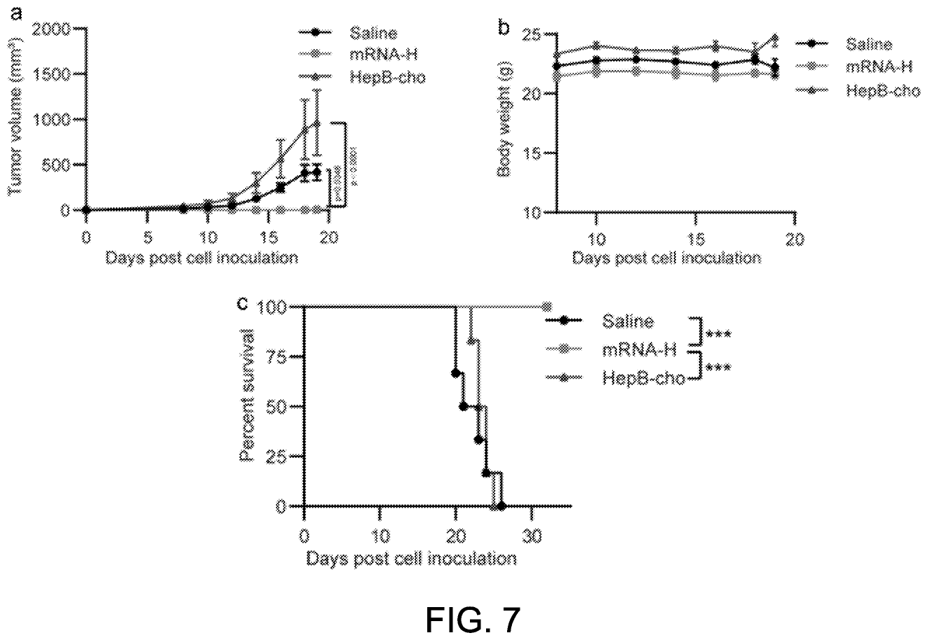

As shown in FIG. 7, after completion of treatment, all mice in the mRNA-H group were tumor-free, while an average tumor volume in the blank group and the HepB-CHO-immunized mice exceeded 1,000 mm³. In addition, no significant change in body weight was observed in mRNA-H-treated mice. All mice in the control group died on Day 26 due to excessive tumor burden, while all mice in the mRNA vaccine-immunized group were still alive on Day 33 and exhibited no tumor growth.

The therapeutic effect of memory T cells generated by the mRNA-H vaccine against B16-HBsAg tumors was verified using a B16-HBsAg tumor model. The results showed that 100% of mice immunized with the mRNA-H vaccine prevented the growth of B16-HBsAg tumors, while the control group was 0%. Furthermore, no significant change in body weight of the mice in the mRNA vaccine group was observed, indicating good safety. The results demonstrate that the HBsAg-specific memory T cells can completely eliminate B16-HBsAg tumors, thereby providing a strong guarantee for subsequent killing of tumors expressing neoantigens derived from heterologous proteins.

### Example 5

### Evaluation of Immunogenicity of the Neoantigen HBsAg

6-week-old female C57BL/6 mice were divided into three groups: 1.B16-HBsAg group, 2. B16-OVA group, and 3. B16-Neo group. The mice were immunized with mRNA-H, mRNA-OVA, and mRNA-Neo, respectively. Immunizations were performed twice, and one week after the final immunization, the mice were inoculated with B16-HBsAg, B16-OVA, and B16F10 cells, the mice was observed for their conditions, and a tumor volume was measured.

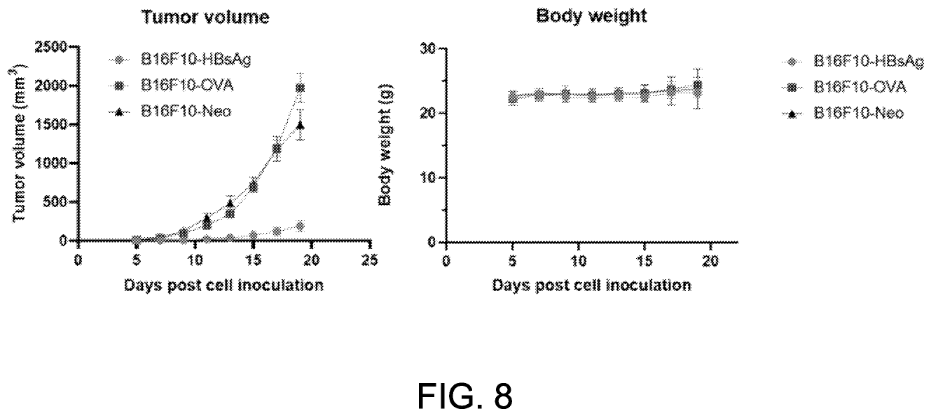

As shown in FIG. 8, the B16-HBsAg group significantly inhibited tumor growth, indicating that mRNA-H can effectively activate memory T cells to kill tumors by marking the HBsAg neoantigen. This also demonstrates that heterologous proteins derived from microorganisms have higher immunogenicity than neoantigens and other highly immunogenic proteins, and can be rapidly recognized and killed by memory cells.

### Example 6

### Evaluation of Immunogenicity of the Neoantigen HBsAg

According to the method described in Example 5, when an average tumor volume of the B16-Neo group reached 2,000 mm³, spleens of the mice in the B16-HBsAg group were removed under sterile conditions and prepared into single-cell suspensions. The suspensions were centrifuged at 350 g for 5 min. Supernatant was discarded, 1 mL of red blood cell lysis buffer was added and lysed for 10 min, followed by addition of 3 mL of buffer to terminate red blood cell lysis. Cells were collected by centrifugation at 350 g for 5 min. 4 mL of RPMI-1640 medium was added to resuspend the cells, which were then counted and adjusted to 2×10⁶/mL. ELISPOT plating was performed as follows: Negative control group: 100 μL cells+100 μL PBS; HBsAg protein group: 100 μL cells+100 μL HBsAg epitope peptide pool; M30 group: 100 μL cells+100 μL M30 peptide; M27 group: 100 μL cells+100 μL M27 peptide. After addition, the plate was covered and incubated for 16-24 h. After incubation, spots were detected and statistically analyzed.

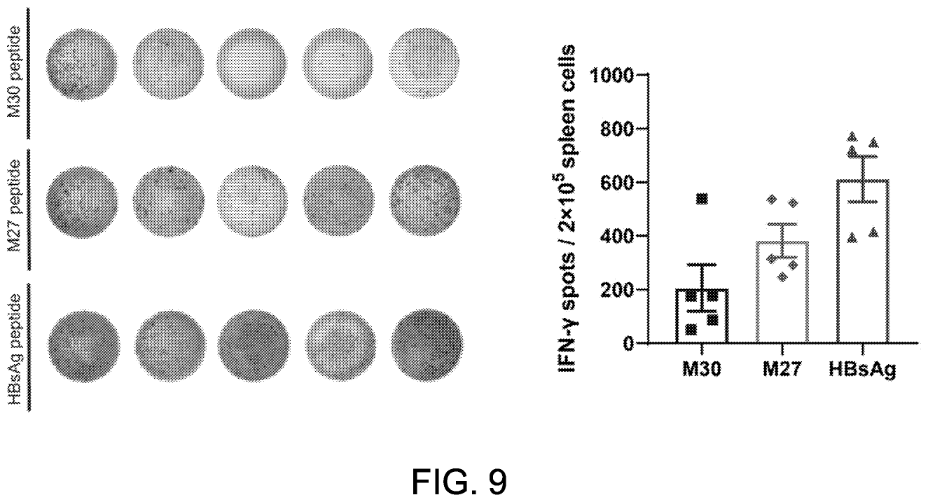

As shown in FIG. 9, tumors marked with the HBsAg neoantigen induced and expanded immune responses to secondary antigens under the continuous killing effect of memory T cells, effectively achieving epitope spreading and contributed to the overall therapeutic outcome.

### Example 7

Construction and characterization of recombinant oncolytic virus expressing neoantigen:

### (1) Construction and Synthesis of HBsAg Shuttle Plasmid

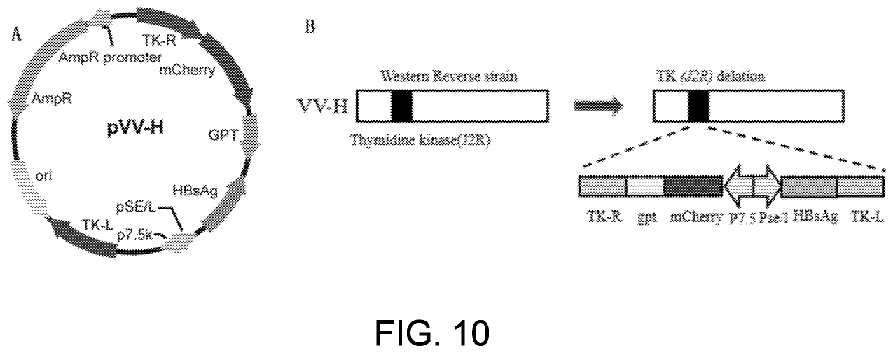

Flanking sequences VACWR093 (TK-R) and VACWR095 (TK-L) of TK (VACWR094) were used as homologous arms; mCherry (fluorescent group) and xanthine-guanine phosphoribosyltransferase (gpt) were used as selection genes; a Pse/l promoter was used to induce HBsAg expression; a vector resistance was AmpR (ampicillin), and a pVV-mcherry-HBsAg plasmid was constructed. In addition, an empty vaccinia virus VV-C and a vaccinia virus VV-G expressing GM-CSF (granulocyte-macrophage colony-stimulating factor) (HBsAg located at the same position in the plasmid shown in the figure) were constructed and synthesized by GenScript Biotech Corporation. A plasmid map is shown in FIG. 10. A nucleotide sequence of the recombinant viral plasmid is shown in SEQ ID NO. 2.

### (2) Construction and Screening of Recombinant Oncolytic Virus (VV-H) Expressing HBsAg

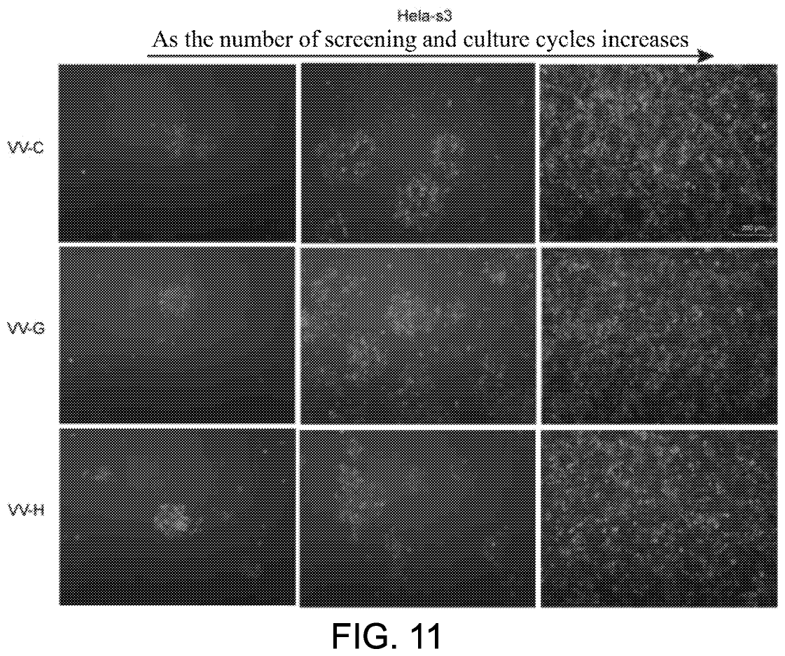

293T cells were cultured, digested with trypsin, and counted; 5×10⁵ cells/well were seeded in 6-well plates and cultured at 37° C. with 5% CO₂. When the cells reached 90% confluence, a wild-type virus (VV) was added at a multiplicity of infection (MOI) of 1. After 2 h of infection, the Dulbecco's modified eagle medium (DMEM) was replaced and 1 μg plasmid/well was transfected. 24-72 h after transfection, red fluorescence was observed and photographed for record. The culture medium was discarded, 500 μL of DMEM without FBS was added and pipetted, and the resulting cells were collected into centrifuge tubes. After repeated freeze-thaw at-80/37° C. for 3 cycles, the cells were then centrifuged at 2,000 rpm for 10 min, and a supernatant was collected. Hela-S3 cells were seeded in 6-well plates. When the confluence reached 90%, 10 and 100 μL of virus were added to three wells, respectively. After 2 h, the culture medium was removed, and a selection medium containing xanthine, hypoxanthine, and mycophenolic acid was added for screening culture. mCherry fluorescence was observed within 24-72 h after adding the selection medium, and fluorescence images are shown in FIG. 11. After 72 h, the selection medium was carefully discarded, 500 μL DMEM was added, centrifugation was performed to collect a supernatant, and the previous steps were repeated. The recombinant virus was screened for three to four rounds, and the screening results are shown in FIGS. 11.

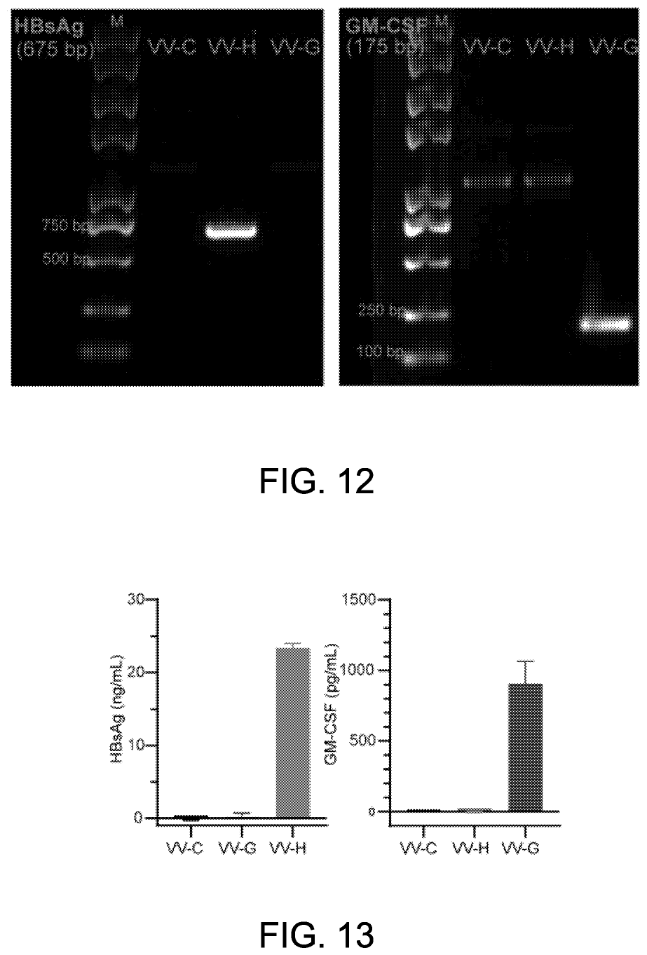

10 and 100 μL of the screened virus were taken to infect cells in two 6-well plates, respectively. DNA was extracted from these cells, and PCR identification was performed using target gene primers to determine whether recombinant virus existed. The identification results are shown in FIG. 12. In addition, an ELISA kit was used to identify the target protein in cell lysate, as shown in FIG. 13. VV-H and VV-G could promote the expression of HBsAg and GM-CSF in tumor cells, respectively. After correct identification at genomic DNA and protein levels, the recombinant virus was amplified using 1× selection medium.

### (3) Plaque Purification of HBsAg Recombinant Oncolytic Virus (VV-H)

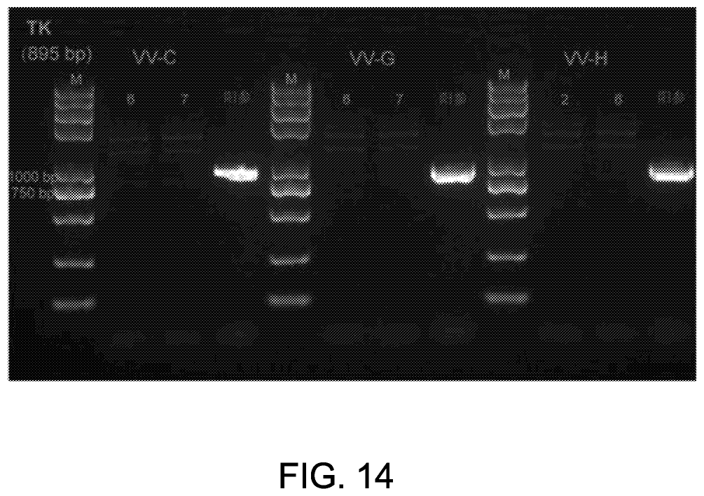

HELA-S3 cell seeding and viral infection followed the same steps as described above. Different volumes of viral solution were added to 1× selection medium for plaque purification to remove wild-type virus. Fluorescence was observed after 24 h. In plates with an appropriate number of plaques, the medium was replaced with 2× selection medium (1 mL), and 1 mL of 2% low-melting-point agarose was added, followed by incubation at 37° C. in a 5% CO₂ incubator. According to the fluorescence observed at 24 h, HELA-S3 cells were seeded in 24-well plates, enabling the cell confluence to reach 90% at 48 h (24-well plate, 10 wells, 5×10⁴ cells/well, 500 μL DMEM containing 2% FBS). At 48 h, a single plaque was picked up using a 200 μL sterile pipette tip and placed in a 24-well plate, and fluorescent plaque formation was continuously observed until the cells were completely confluent. A supernatant was removed, 200 μL FBS-free DMEM was added, pipetted repeatedly, and centrifuged to collect the virus in the supernatant, with the steps the same as above. Thymidine kinase (TK) primers were used to identify whether the selected recombinant virus contained wild-type virus, until no wild-type virus was identified in the solution. As shown in FIG. 14, the selected recombinant virus no longer contained wild-type virus.

### (4) In Vitro Amplification and Purification of Recombinant Oncolytic Virus VV-H

Multiple dishes of Hela-S3 (cervical cancer) cells were prepared, digested, and counted to approximately 10⁸ cells. The above recombinant virus (MOI=0.1) and the cell mixture were seeded into 20 culture dishes of 15 cm for amplification of the recombinant virus. Fluorescence expression was observed during amplification. When fluorescence intensity reached a highest level, the cells and virus solution were transferred to 50 mL centrifuge tubes, and centrifuged at 2,000 g for 10 min; the supernatant was discarded, and 10 mM Tris-HCl was added to resuspend the cells. The above cell suspension was subjected to repeated freeze-thaw at −80/37° C. for 3 cycles, centrifuged at 2,000 rpm for 5 min, and the supernatant was collected. 1/3 volume of trypsin was added to the supernatant, mixed evenly, and incubated in a 37° C. water bath for 30 min. Preparation of 36% sucrose: 13 mL of 36% sucrose was added into an ultracentrifuge tube, and an equal volume of viral solution was gently added to a top layer of the sucrose solution. Centrifugation was performed at 32,900 g for 2 h at 4° C. The supernatant was gently discarded, and each ultracentrifuge tube was resuspended with 5 mL PBS; PBS was added to fill up the tube. Centrifugation was performed again at 32,900 g for 1 h at 4° C. The supernatant was discarded, 1 mL PBS was added to each ultracentrifuge tube to resuspend, the viral solution was aliquoted and stored at −80° C. to obtain recombinant vaccinia virus.

### Titer Determination (TCID₅₀) of Recombinant Vaccinia Virus

Hela-S3 cells were seeded into 96-well plates at a density of 10⁴ cells/well, and serial viral dilutions of 10⁻³, 10⁻⁴, 10⁻⁵, 10⁻⁶, 10⁻⁷, 10⁻⁸, 1⁻⁹, and 10⁻¹⁰ were prepared, with 6 replicates for each viral dilution and 3 replicates for each virus. Fluorescent plaques in each well were observed. A viral titer was calculated according to the formula T=0.7×10×10^{(1+S(d-0.5))}, where S=log dilution−1, and d=A sum of positive ratios.

### Detection of the Ability of VV-H to Mark HBsAg by ELISA Kit

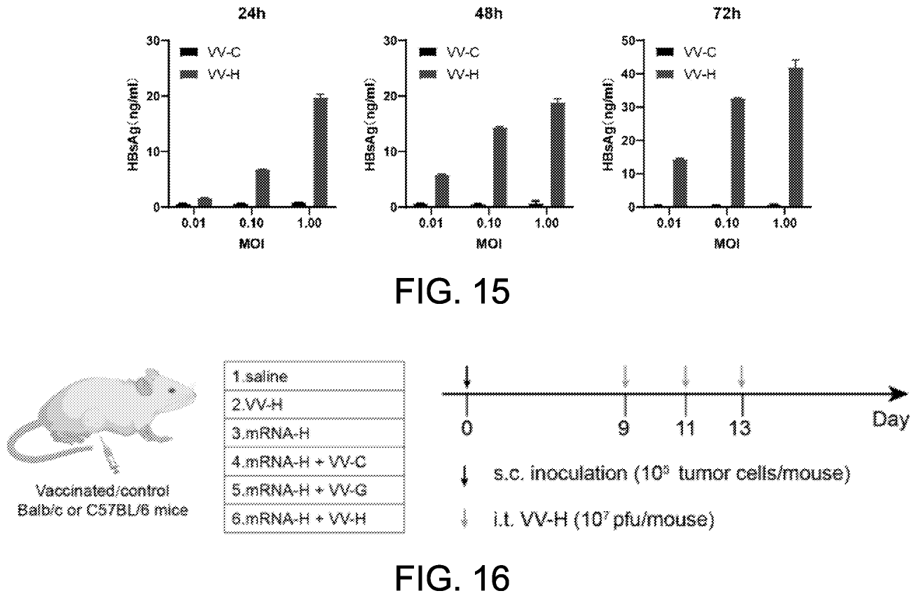

Hela-S3 tumor cells were infected with virus at MOIs of 0.01, 0.1, and 1. Cells were collected by centrifugation at 24, 48, and 72 h after infection. After repeated freeze-thaw at −80/37° C. for 3 cycles, centrifugation was performed at 2,000 rpm for 10 min, and the supernatant was collected. HBsAg content was detected by using an ELISA kit and the absorbance at OD450 nm was read using a microplate reader. The expression results are shown in FIG. 15.

### Example 8

### Evaluation of Tumor Therapeutic Effect Produced by Activation of its Specific Memory T Cells by VV-H

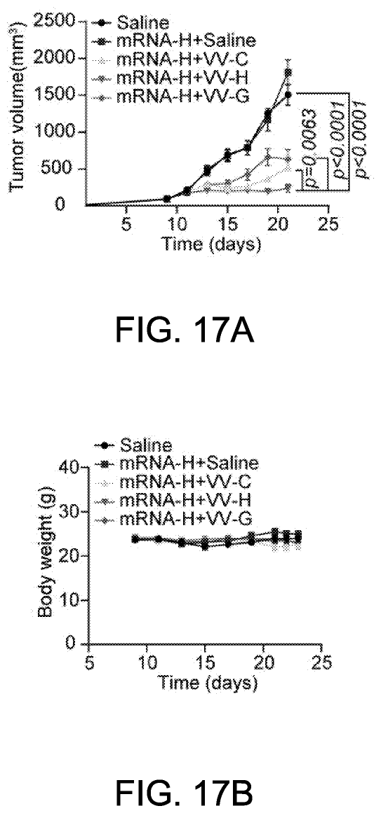

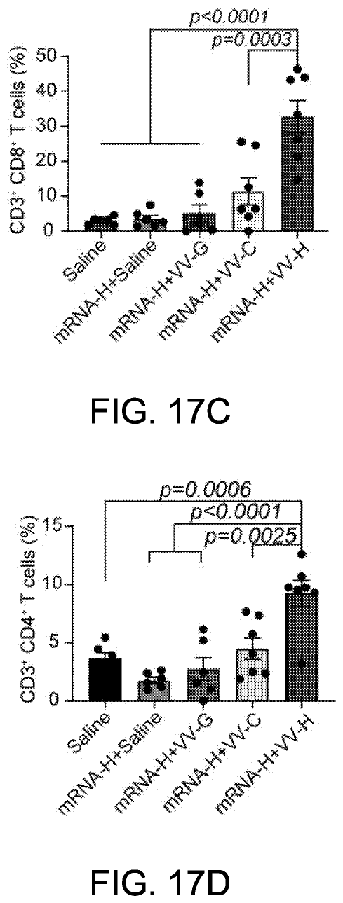

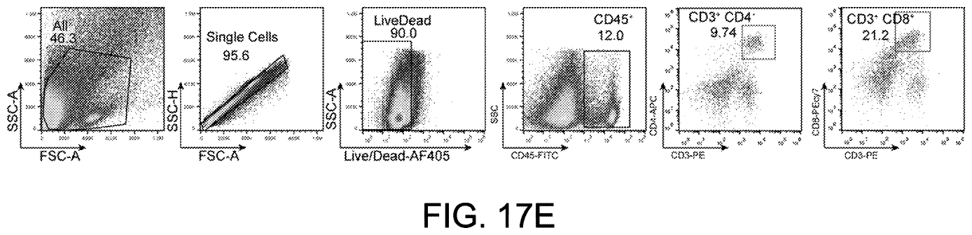

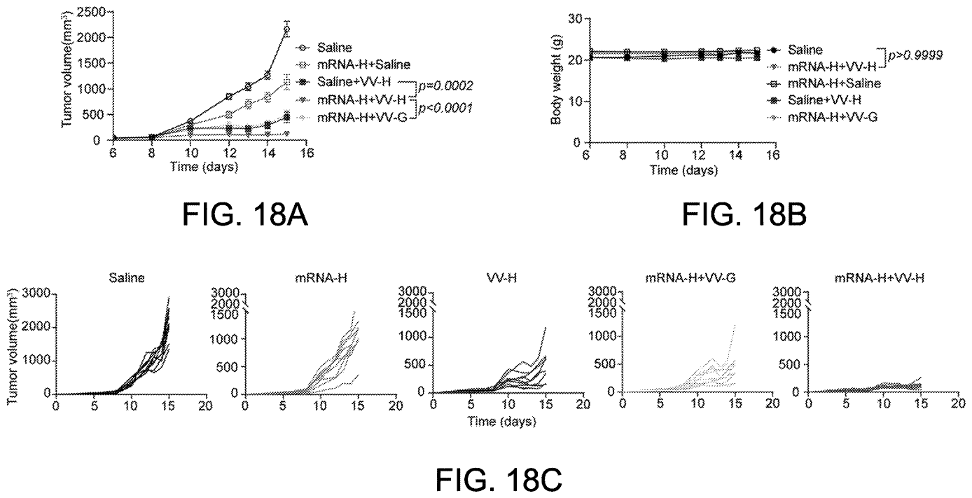

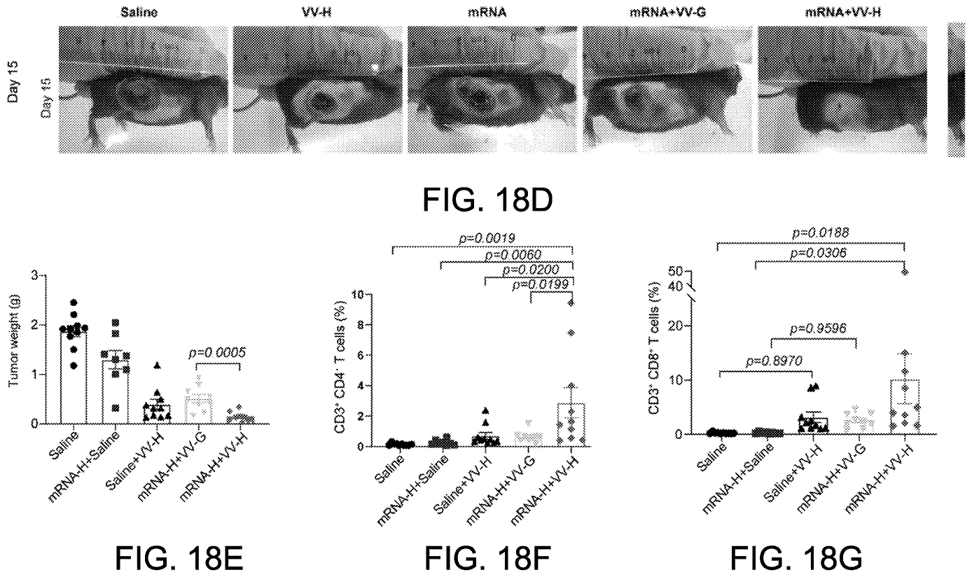

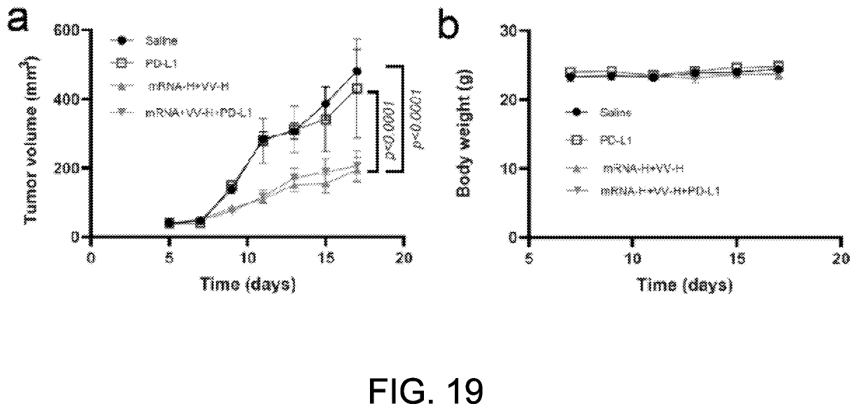

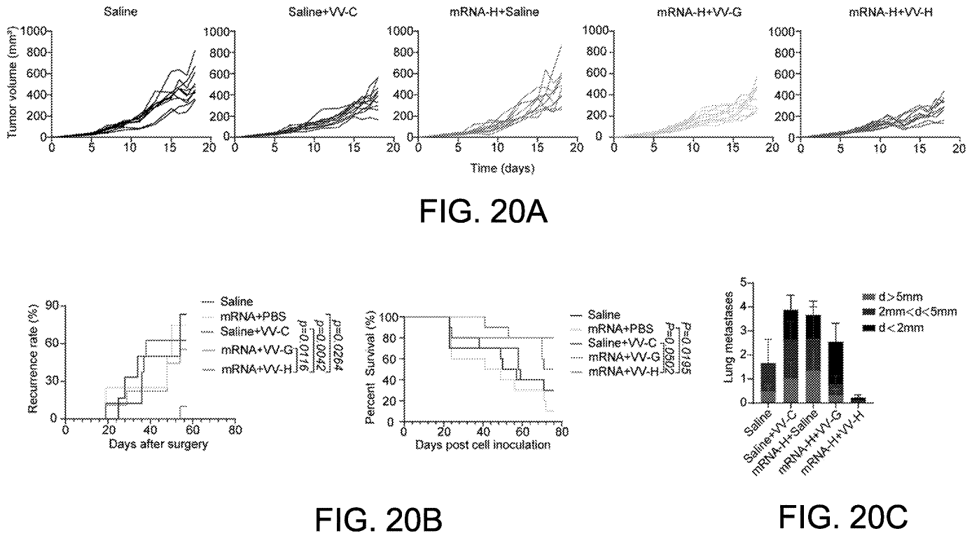

The therapeutic effect of treatment was evaluated using four tumor models, including B16F10 melanoma (rapid growth rate and high malignancy), CT26 colorectal cancer (strong tumor heterogeneity and immunologically cold tumor), 4T1 breast cancer (low mutation burden and spontaneous metastasis), and B16-OVA. The specific treatment method is shown in FIG. 16: a control group and C57BL/6 or Balb/c mice 30 days after immunization were used, and tumor cells were subcutaneously inoculated to establish B16F10, CT26, and 4T1 tumor models, respectively. When a tumor volume reached 50-100 mm³, these tumor-bearing mice were randomly divided into 5 groups. On Day 0 (the day of grouping), Day 3, and Day 5, the mice was intratumorally injected with 50 μL of each virus solution (107 PFU/mouse). The results are shown in FIGS. 17A-20C (FIGS. 17A to 17E: B16F10 subcutaneous tumor model; FIGS. 18A to 18G: B16-OVA subcutaneous tumor model; FIG. 19: CT26 subcutaneous tumor model; FIGS. 20A to 20C: 4T1 subcutaneous tumor model). Compared with the control group, VV-H+ mRNA-H significantly inhibited tumors, indicating that VV-H+ mRNA-H can effectively activate memory T cells to kill tumors by marking the HBsAg neoantigen. In addition, no significant change in body weight was observed in each group of mice, indicating that the treatment exhibited good safety.

Furthermore, in the 4T1 tumor model, the tumor on the treated side was resected 30 days after the last treatment, and the body weight change of the mice was monitored. At the treatment endpoint, survival curves and lung metastasis statistically analyzed, as shown in FIGS. 20A to 20C, this treatment strategy can effectively slow down the ability of tumors to metastasize to distant sites.

### Example 9

### Evaluation of Melanoma Therapeutic Effect Produced by Activation of HBsAg Memory T Cells by VV-H

After completion of treatment in the B16F10 melanoma model and B16-OVA subcutaneous tumor model in Example 7, the tumors and spleens on the treated side were dissected, and single-cell suspensions were prepared. Subsequently, the single-cell suspensions of tumor were stained on ice for 30 min with a mixture of CD45, CD3, CD4, and CD8 antibodies and Live/Dead dye, and a content of effector T cells was detected by flow cytometry.

As shown in FIGS. 17A to 17E, in the B16F10 melanoma model, the VV-H+ mRNA-H group showed at least a 10-fold increase in CD8⁺ T cells and at least a 4-fold increase in CD4⁺ T cells in tumors of mice compared with the mRNA-H vaccine group, indicating that VV-H+mRNA-H efficiently lyses tumors, releases antigens, and further activates anti-tumor immune response.

Similarly, as shown in FIGS. 18A to 18G, in the B16-OVA subcutaneous tumor model, the VV-H+ mRNA-H group showed a significant increase in CD4+ and CD8⁺ T cells in tumors of mice compared with other treatment groups, indicating that the VV-H+ mRNA-H group achieved more effective immune infiltration.

### Example 10

### Evaluation of the Ability of VV-H to Induce In Vivo Tumor Expression of HBsAg Using Immunofluorescence Staining

A B16F10 tumor model was constructed by subcutaneously inoculating B16F10 cells into a control group and immunized C57BL/6 mice. When a tumor volume reached 150 mm³, the mice were randomly divided into three groups (n=5): 1. VV-C group; 2. VV-H group; and 3. mRNA-H+VV-H group. After grouping, 50 μL of virus (107 PFU/mouse) was intratumorally injected. On Days 1, 3, and 7 after administration, tumors were dissected, sectioned, and observed for VV-H (mCherry, red) and HBsAg neoantigen (FITC-HBsAg antibody, green).

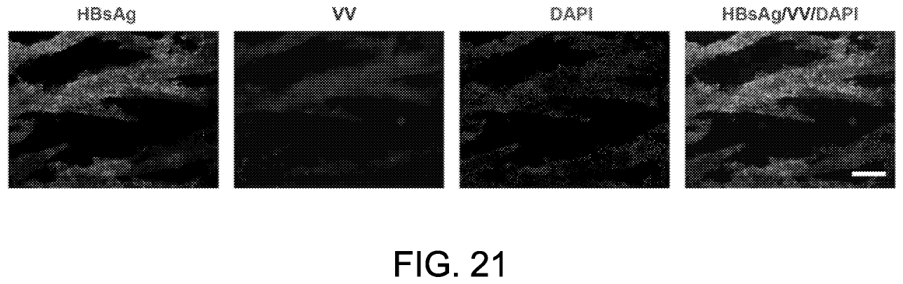

As shown in FIG. 21, after VV-H administration, a strong HBsAg signal could be detected in tumors, indicating that VV-H can effectively mark HBsAg.

### Example 11

Safety Evaluation of VV-H+ mRNA-H Treatment

In the above treatment process, the body weight of the mouse was monitored daily to preliminarily evaluate safety. In addition, according to the same treatment method as described in Example 8, on Day 1 and Day 14 after treatment, major hematological indicators such as red blood cells and white blood cells, as well as serum biochemical indicators such as alanine aminotransferase (ALT) and blood urea nitrogen (BUN), were detected. After completion of treatment, the mice were euthanized, and the heart, liver, spleen, lung, and kidney were separated for pathological examination to further evaluate safety.

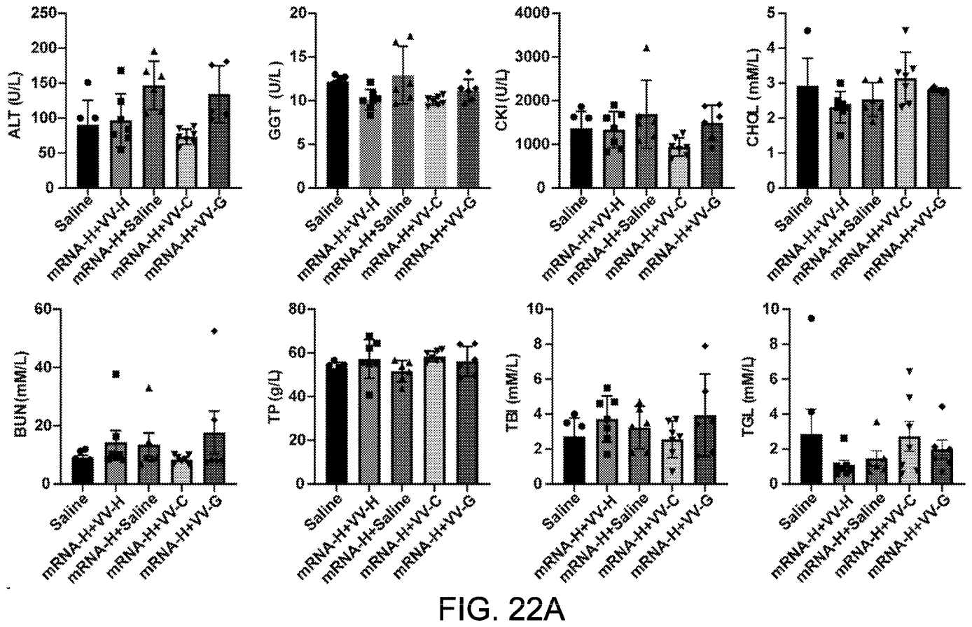

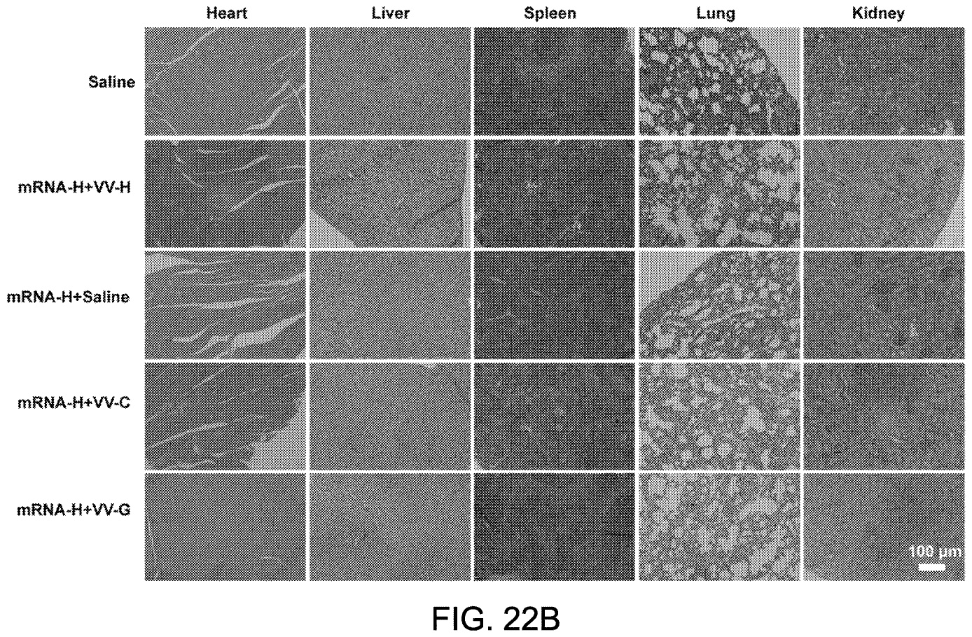

As shown in FIGS. 22A and 22B, no significant changes were observed in serum biochemical indicators or HE staining of major organs in mice in the VV-H+ mRNA-H group, preliminarily demonstrating good safety.

What is claimed is:

1. A tumor neoantigen vaccine system configured for marking a heterologous protein, comprising a heterologous protein mRNA vaccine and a recombinant oncolytic virus, wherein the heterologous protein mRNA vaccine generates specific memory T cells that configured to mark neoantigens, and the recombinant oncolytic virus is configured to mark tumor cells with the neoantigens.

2. The vaccine system according to claim 1, wherein the heterologous protein is one or more selected from Hepatitis B virus-derived protein, human papillomavirus-derived protein, EBV-derived protein, influenza virus-derived protein, tetanus toxoid protein, Staphylococcus aureus-derived protein, and flagellin protein of Salmonella yphi.

3. The vaccine system according to claim 1, wherein the oncolytic virus is selected from one or more of vaccinia virus, herpes simplex virus, and adenovirus.

4. The vaccine system according to claim 1, wherein the heterologous protein mRNA vaccine is prepared by constructing, synthesizing, and amplifying a recombinant plasmid of the heterologous protein mRNA vaccine, followed by encapsulating the recombinant plasmid of the heterologous protein mRNA vaccine with lipid nanoparticles.

5. The vaccine system according to claim 1, wherein the recombinant oncolytic virus is prepared by constructing an oncolytic virus expressing the neoantigen, followed by plaque purification and amplification purification.

6. A use of the vaccine system according to claim 1 in tumor immunotherapy.

7. The use according to claim 6, wherein the vaccine system is used to enhance tumor immunotherapy.

8. The use according to claim 6, wherein an administration sequence is as follows: administering the heterologous protein mRNA vaccine, followed by administering the recombinant oncolytic virus.

9. The use according to claim 8, wherein the heterologous protein mRNA vaccine is administered by intramuscular injection, intranasal administration, or intravenous injection to induce immune memory, and the recombinant oncolytic virus is administered intratumorally to mark the tumor cells with the neoantigen.

10. The use according to claim 6, wherein the tumor is a solid tumor or a solid tumor with concomitant metastasis, comprising melanoma, colorectal cancer, breast cancer, liver cancer, glioblastoma, cholangiocarcinoma, or pancreatic cancer.


SEQUENCE LISTING (2 sequences)

SEQ ID NO: 1 - protein, 3481 aa, synthetic construct
TCGCGCGTTTCGGTGATGACGGTGAAAACCTCTGACACATGCAGCTCCCGGAGACTGTCACAGCTTGTCTGTAAGCGGATGCCGGGAGCAGACAAGCCCGTCAGGGCGCGTCAGCGGGTGTTGGCGGGTGTCGGGGCTGGCTTAACTATGCGGCATCAGAGCAGATTGTACTGAGAGTGCACCATATGCGGTGTGAAATACCGCACAGATGCGTAAGGAGAAAATACCGCATCAGGCGCCATTCGCCATTCAGGCTGCGCAACTGTTGGGAAGGGCGATCGGTGCGGGCCTCTTCGCTATTACGCCAGCTGGCGAAAGGGGGATGTGCTGCAAGGCGATTAAGTTGGGTAACGCCAGGGTTTTCCCAGTCACGACGTTGTAAAACGACGGCCAGTGAATTGACGCGTATTGGTAATACGACTCACTATAAGAATAAACTAGTATTCTTCTGGTCCCCACAGACTCAGAGAGAACCCGCCACCATGGAGAACATCACAAGCGGCTTCCTGGGCCCTCTCCTGGTGCTGCAAGCCGGCTTCTTCCTGCTGACAAGGATCCTGACCATCCCTCAGAGCCTGGACAGCTGGTGGACAAGCCTGAACTTCCTGGGCGGCACCACCGTGTGCCTGGGACAGAACAGCCAAAGCCCTACAAGCAACCACTCCCCCACAAGCTGTCCTCCTACCTGCCCTGGCTATAGGTGGATGTGCCTGAGAAGGTTCATCATCTTCCTGTTCATCCTGCTCCTGTGCCTGATCTTCCTGCTGGTGCTGCTGGACTACCAAGGCATGCTGCCTGTGTGCCCTCTGATCCCTGGCAGCAGCACCACAAGCACCGGCCCTTGTAGGACCTGCATGACCACCGCCCAAGGCACAAGCATGTACCCTAGCTGCTGTTGCACCAAGCCTAGCGACGGCAACTGCACCTGCATCCCTATCCCTAGCAGCTGGGCCTTCGGCAAGTTCCTGTGGGAGTGGGCTAGCGCTAGGTTCAGCTGGCTGAGCCTGCTGGTGCCTTTCGTGCAGTGGTTCGTGGGCCTGAGCCCTACCGTGTGGCTGAGCGTGATCTGGATGATGTGGTACTGGGGCCCTAGCCTGTACAGCATCCTGAGCCCTTTCCTGCCTCTGCTGCCTATCTTCTTCTGCCTGTGGGTGTACATCTGAGCTGGAGCCTCGGTGGCCTAGCTTCTTGCCCCTTGGGCCTCCCCCCAGCCCCTCCTCCCCTTCCTGCACCCGTACCCCCGTGGTCTTTGAATAAAGTCTGAGTGGGCGGCCCAATGGCGCGCCGAGCTTGGCTCGAGCATGGTCATAGCTGTTTCCTGTGTGAAATTGTTATCCGCTCACAATTCCACACAACATACGAGCCGGAAGCATAAAGTGTAAAGCCTGGGGTGCCTAATGAGTGAGCTAACTCACATTAATTGCGTTGCGCTCACTGCCCGCTTTCCAGTCGGGAAACCTGTCGTGCCAGCTGCATTAATGAATCGGCCAACGCGCGGGGAGAGGCGGTTTGCGTATTGGGCGCTGTTCCGCTTCCTCGCTCACTGACTCGCTGCGCTCGGTCGTTCGGCTGCGGCGAGCGGTATCAGCTCACTCAAAGGCGGTAATACGGTTATCCACAGAATCAGGGGATAACGCAGGAAAGAACATGTGAGCAAAAGGCCAGCAAAAGGCCAGGAACCGTAAAAAGGCCGCGTTGCTGGCGTTTTTCCATAGGCTCCGCCCCCCTGACGAGCATCACAAAAATCGACGCTCAAGTCAGAGGTGGCGAAACCCGACAGGACTATAAAGATACCAGGCGTTTCCCCCTGGAAGCTCCCTCGTGCGCTCTCCTGTTCCGACCCTGCCGCTTACCGGATACCTGTCCGCCTTTCTCCCTTCGGGAAGCGTGGCGCTTTCTCATAGCTCACGCTGTAGGTATCTCAGTTCGGTGTAGGTCGTTCGCTCCAAGCTGGGCTGTGTGCACGAACCCCCCGTTCAGCCCGACCGCTGCGCCTTATCCGGTAACTATCGTCTTGAGTCCAACCCGGTAAGACACGACTTATCGCCACTGGCAGCAGCCACTGGTAACAGGATTAGCAGAGCGAGGTATGTAGGCGGTGCTACAGAGTTCTTGAAGTGGTGGCCTAACTACGGCTACACTAGAAGAACAGTATTTGGTATCTGCGCTCTGCTGAAGCCAGTTACCTTCGGAAAAAGAGTTGGTAGCTCTTGATCCGGCAAACAAACCACCGCTGGTAGCGGTGGTTTTTTTGTTTGCAAGCAGCAGATTACGCGCAGAAAAAAAGGATCTCAAGAAGATCCTTTGATCTTTTCTACGGGGTCTGACGCTCAGTGGAACGAAAACTCACGTTAAGGGATTTTGGTCATGAGATTATCAAAAAGGATCTTCACCTAGATCCTTTTAAATTAAAAATGAAGTTTTAAATCAATCTAAAGTATATATGAGTAAACTTGGTCTGACAGTTAGAAAAACTCATCGAGCATCAAATGAAACTGCAATTTATTCATATCAGGATTATCAATACCATATTTTTGAAAAAGCCGTTTCTGTAATGAAGGAGAAAACTCACCGAGGCAGTTCCATAGGATGGCAAGATCCTGGTATCGGTCTGCGATTCCGACTCGTCCAACATCAATACAACCTATTAATTTCCCCTCGTCAAAAATAAGGTTATCAAGTGAGAAATCACCATGAGTGACGACTGAATCCGGTGAGAATGGCAAAAGTTTATGCATTTCTTTCCAGACTTGTTCAACAGGCCAGCCATTACGCTCGTCATCAAAATCACTCGCATCAACCAAACCGTTATTCATTCGTGATTGCGCCTGAGCGAAACGAAATACGCGATCGCTGTTAAAAGGACAATTACAAACAGGAATCGAATGCAACCGGCGCAGGAACACTGCCAGCGCATCAACAATATTTTCACCTGAATCAGGATATTCTTCTAATACCTGGAATGCTGTTTTCCCAGGGATCGCAGTGGTGAGTAACCATGCATCATCAGGAGTACGGATAAAATGCTTGATGGTCGGAAGAGGCATAAATTCCGTCAGCCAGTTTAGTCTGACCATCTCATCTGTAACATCATTGGCAACGCTACCTTTGCCATGTTTCAGAAACAACTCTGGCGCATCGGGCTTCCCATACAATCGATAGATTGTCGCACCTGATTGCCCGACATTATCGCGAGCCCATTTATACCCATATAAATCAGCATCCATGTTGGAATTTAATCGCGGCCTAGAGCAAGACGTTTCCCGTTGAATATGGCTCATACTCTTCCTTTTTCAATATTATTGAAGCATTTATCAGGGTTATTGTCTCATGAGCGGATACATATTTGAATGTATTTAGAAAAATAAACAAATAGGGGTTCCGCGCACATTTCCCCGAAAAGTGCCACCTGACGTCTAAGAAACCATTATTATCATGACATTAACCTATAAAAATAGGCGTATCACGAGGCCCTTTTGTC

SEQ ID NO: 2 - protein, 5205 aa, synthetic construct
GACGTCAGCTTTTGCGATCAATAAATGGATCACAACCAGTATCTCTTAACGATGTTCTTCGCAGATGATGATTCATTTTTTAAGTATTTGGCTAGTCAAGATGATGAATCTTCATTATCTGATATATTGCAAATCACTCAATATCTAGACTTTCTGTTATTATTATTGATCCAATCAAAAAATAAATTAGAAGCCGTGGGTCATTGTTATGAATCTCTTTCAGAGGAATACAGACAATTGACAAAATTCACAGACTTTCAAGATTTTAAAAAACTGTTTAACAAGGTCCCTATTGTTACAGATGGAAGGGTCAAACTTAATAAAGGATATTTGTTCGACTTTGTGATTAGTTTGATGCGATTCAAAAAAGAATCCTCTCTAGCTACCACCGCAATAGATCCTGTTAGATACATAGATCCTCGTCGCAATATCGCATTTTCTAACGTGATGGATATATTAAAGTCGAATAAAGTGAACAATAATTAATTCTTTATTGTCATCATGGTGAGCAAGGGCGAGGAGGATAACATGGCCATCATCAAGGAGTTCATGCGCTTCAAGGTGCACATGGAGGGCTCCGTGAACGGCCACGAGTTCGAGATCGAGGGCGAGGGCGAGGGCCGCCCCTACGAGGGCACCCAGACCGCCAAGCTGAAGGTGACCAAGGGTGGCCCCCTGCCCTTCGCCTGGGACATCCTGTCCCCTCAGTTCATGTACGGCTCCAAGGCCTACGTGAAGCACCCCGCCGACATCCCCGACTACTTGAAGCTGTCCTTCCCCGAGGGCTTCAAGTGGGAGCGCGTGATGAACTTCGAGGACGGCGGCGTGGTGACCGTGACCCAGGACTCCTCCCTGCAGGACGGCGAGTTCATCTACAAGGTGAAGCTGCGCGGCACCAACTTCCCCTCCGACGGCCCCGTAATGCAGAAGAAGACCATGGGCTGGGAGGCCTCCTCCGAGCGGATGTACCCCGAGGACGGCGCCCTGAAGGGCGAGATCAAGCAGAGGCTGAAGCTGAAGGACGGCGGCCACTACGACGCTGAGGTCAAGACCACCTACAAGGCCAAGAAGCCCGTGCAGCTGCCCGGCGCCTACAACGTCAACATCAAGTTGGACATCACCTCCCACAACGAGGACTACACCATCGTGGAACAGTACGAACGCGCCGAGGGCCGCCACTCCACCGGCGGCATGGACGAGCTGTACAAGGCAACAAACTTCTCTCTGCTGAAACAAGCCGGAGATGTCGAAGAGAATCCTGGACCGATGAGCGAAAAATACATCGTCACCTGGGACATGTTGCAGATCCATGCACGTAAACTCGCAAGCCGACTGATGCCTTCTGAACAATGGAAAGGCATTATTGCCGTAAGCCGTGGCGGTCTGGTACCGGGTGCGTTACTGGCGCGTGAACTGGGTATTCGTCATGTCGATACCGTTTGTATTTCCAGCTACGATCACGACAACCAGCGCGAGCTTAAAGTGCTGAAACGCGCAGAAGGCGATGGCGAAGGCTTCATCGTTATTGATGACCTGGTGGATACCGGTGGTACTGCGGTTGCGATTCGTGAAATGTATCCAAAAGCGCACTTTGTCACCATCTTCGCAAAACCGGCTGGTCGTCCGCTGGTTGATGACTATGTTGTTGATATCCCGCAAGATACCTGGATTGAACAGCCGTGGGATATGGGCGTCGTATTCGTCCCGCCAATCTCCGGTCGCTAACAGGTGATCTGTTTTTATTGTGGAGATAAAAATTAATTAAATGTATACCCAAAGACAAAAGAAAATTGGTAACAGCGGTAAAAAGGGACTCAAGATGCTGTACAGACTTGGCCCCCAATACCACATCATCCATATAACTGAAAGCCAAACAGTGGGGGAAAGCCCTACGAACCACTGAACAAATGGCACTAGTAAACTGAGCCAGGAGAAACGGGCTGAGGCCCACTCCCATAGGAATTTTCCGAAAGCCCAGGATGATGGGATGGGAATACAGGTGCAATTTCCGTCCGAAGGTTTGGTACAGCAACAGGAGGGATACATAGAGGTTCCTTGAGCAGTAGTCATGCAGGTCCGGCATGGTCCCGTGCTGGTTGTTGAGGATCCTGGAATTAGAGGACAAACGGGCAACATACCTTGATAGTCCAGAAGAACCAACAAGAAGATGAGGCATAGCAGCAGGATGAAGAGGAAGATGATAAAACGCCGCAGACACATCCAGCGATAACCAGGACAAGTTGGAGGACAAGAGGTTGGTGAGTGATTGGAGGTTGGGGACTGCGAATTTTGGCCAAGACACACGGTAGTTCCCCCTAGAAAATTGAGAGAAGTCCACCACGAGTCTAGACTCTGCGGTATTGTGAGGATTCTTGTCAACAAGAAAAACCCCGCCTGTAACACGAGAAGGGGTCCTAGGAATCCTGATGTGATGTTCTCCATGGTGGCGGCCGCTCGAGCTTATTTATATTCCAAAAAAAAAAAATAAAATTTCAATTTTTAAGCTTTCACTAATTCCAAACCCACCCGCTTTTTATAGTAAGTTTTTCACCCATAAATAATAAATACAATAATTAATTTCTCGTAAAAGTAGAAAATATATTCTAATTTATTGCACGGTAAGGAAGTAGAATCATAAAGAACAGTACTCAATCAATAGCAATCATGAAACAATATATCGTCCTGGCGCGCCGAGCTCGGATCCTATTATATTTTTTATCTAAAAAACTAAAAATAAACATTGATTAAATTTTAATATAATACTTAAAAATGGATGTTGTGTCGTTAGATAAACCGTTTATGTATTTTGAGGAAATTGATAATGAGTTAGATTACGAACCAGAAAGTGCAAATGAGGTCGCAAAAAAACTGCCGTATCAAGGACAGTTAAAACTATTACTAGGAGAATTATTTTTTCTTAGTAAGTTACAGCGACACGGTATATTAGATGGTGCCACCGTAGTGTATATAGGATCTGCTCCCGGTACACATATACGTTATTTGAGAGATCATTTCTATAATTTAGGAGTGATCATCAAATGGATGCTAATTGACGGCCGCCATCATGATCCTATTTTAAATGGATTGCGTGATGTGACTCTAGTGACTCGGTTCGTTGATGAGGAATATCTACGATCCATCAAAAAACAACTGCATCCTTCTAAGATTATTTTAATTTCTGATGTGAGATCCAAACGAGGAGGAAATGAACCTAGTACGGCGGATTTACTAAGTAATTACGCTCTACAAAATGTCATGATTAGTATTTTAAACCCCGTGGCGTCTAGTCTTAAATGGAGATGCCCGTTTCCAGATCAATGGATCAAGGACTTTTATATCCCACACGGTAATAAAATGTTACAACCTTTTGCTCCTTCATATTCAGCTGAAATGAGATTATTAAGTATTTATACCGGTGAGAACATGAGACTGACTCGGGCCGCGTTGCTGGCGTTTTTCCATAGGCTCCGCCCCCCTGACGAGCATCACAAAAATCGACGCTCAAGTCAGAGGTGGCGAAACCCGACAGGACTATAAAGATACCAGGCGTTTCCCCCTGGAAGCTCCCTCGTGCGCTCTCCTGTTCCGACCCTGCCGCTTACCGGATACCTGTCCGCCTTTCTCCCTTCGGGAAGCGTGGCGCTTTCTCATAGCTCACGCTGTAGGTATCTCAGTTCGGTGTAGGTCGTTCGCTCCAAGCTGGGCTGTGTGCACGAACCCCCCGTTCAGCCCGACCGCTGCGCCTTATCCGGTAACTATCGTCTTGAGTCCAACCCGGTAAGACACGACTTATCGCCACTGGCAGCAGCCACTGGTAACAGGATTAGCAGAGCGAGGTATGTAGGCGGTGCTACAGAGTTCTTGAAGTGGTGGCCTAACTACGGCTACACTAGAAGGACAGTATTTGGTATCTGCGCTCTGCTGAAGCCAGTTACCTTCGGAAAAAGAGTTGGTAGCTCTTGATCCGGCAAACAAACCACCGCTGGTAGCGGTGGTTTTTTTGTTTGCAAGCAGCAGATTACACGCAGAAAAAAAGGATCTCAAGAAGATCCTTTGATCTTTTCTACGGGGTCTGACGCTCAGTGGAACGAAAACTCACGTTAAGGGATTTTGGTCATGAGATTATCAAAAAGGATCTTCACCTAGATCCTTTTAAATTAAAAATGAAGTTTTAAATCAATCTAAAGTATATATGAGTAAACTTGGTCTGACAGTTACCAATGCTTAATCAGTGAGGCACCTATCTCAGCGATCTGTCTATTTCGTTCATCCATAGTTGCCTGACTCCCCGTCGTGTAGATAACTACGATACGGGAGGGCTTACCATCTGGCCCCAGTGCTGCAATGATACCGCGAGACCCACGCTCACCGGCTCCAGATTTATCAGCAATAAACCAGCCAGCCGGAAGGGCCGAGCGCAGAAGTGGTCCTGCAACTTTATCCGCCTCCATCCAGTCTATTAATTGTTGCCGGGAAGCTAGAGTAAGTAGTTCGCCAGTTAATAGTTTGCGCAACGTTGTTGCCATTGCTGCAGGCATCGTGGTGTCACGCTCGTCGTTTGGTATGGCTTCATTCAGCTCCGGTTCCCAACGATCAAGGCGAGTTACATGATCCCCCATGTTGTGCAAAAAAGCGGTTAGCTCCTTCGGTCCTCCGATCGTTGTCAGAAGTAAGTTGGCCGCAGTGTTATCACTCATGGTTATGGCAGCACTGCATAATTCTCTTACTGTCATGCCATCCGTAAGATGCTTTTCTGTGACTGGTGAGTACTCAACCAAGTCATTCTGAGAATAGTGTATGCGGCGACCGAGTTGCTCTTGCCCGGCGTCAACACGGGATAATACCGCGCCACATAGCAGAACTTTAAAAGTGCTCATCATTGGAAAACGTTCTTCGGGGCGAAAACTCTCAAGGATCTTACCGCTGTTGAGATCCAGTTCGATGTAACCCACTCGTGCACCCAACTGATCTTCAGCATCTTTTACTTTCACCAGCGTTTCTGGGTGAGCAAAAACAGGAAGGCAAAATGCCGCAAAAAAGGGAATAAGGGCGACACGGAAATGTTGAATACTCATACTCTTCCTTTTTCAATATTATTGAAGCATTTATCAGGGTTATTGTCTCATGAGCGGATACATATTTGAATGTATTTAGAAAAATAAACAAATAGGGGTTCCGCGCACATTTCCCCGAAAAGTGCCACCT